(19) United States
(12) Patent Application Publication
(10) Pub. No.: US 2015/0169844 A1
(43) Pub. Date: Jun. 18, 2015

(54) DISORDER TREATMENT MANAGEMENT SYSTEM AND METHOD

(71) Applicant: Sleep Data Services, LLC, San Diego, CA (US)

(72) Inventors: Dominic Munafo, San Diego, CA (US); David French, La Jolla, CA (US); Bretton Hevener, San Diego, CA (US); William Hevener, San Diego, CA (US); Thinh Nguyen, San Diego, CA (US)

(21) Appl. No.: 14/547,119

(22) Filed: Nov. 18, 2014

Related U.S. Application Data
(60) Provisional application No. 61/905,775, filed on Nov. 18, 2013.

(51) Int. Cl. G06F 19/00

(52) U.S. Cl. CPC G06F 19/3481; G06F 19/3443

(57) ABSTRACT

A system and method for managing treatment of a disorder of a person are disclosed. Data from multiple sources is collected in a data warehouse by a server computer. The data is associated with the disorder, and the sources including a clinical data source and a real-time monitoring source associated with the person. The collected data is normalized against a population of persons sharing the disorder. The normalized data is then associated with the person to generate a treatment regime of the disorder. A compliance by the person with the treatment regime is then calculated based on operational metrics for the population and the data collected from the plurality of sources.

### CROSS-REFERENCE TO RELATED APPLICATIONS

This application claims priority under 35 U.S.C. §119(e) to U.S. Provisional patent application Ser. No. 61/905,775 filed on Nov. 18, 2013 and entitled “Disorder Treatment Management System and Method” the disclosure of which is incorporated by reference herein in its entirety.

### TECHNICAL FIELD

The subject matter described herein relates to patient management, and more particularly to a system and method for managing treatment of a disorder, which is treated with one or more treatment devices.

### BACKGROUND

As one example, the treatment of sleep apnea has advanced to the point that the devices used to treat sleep apnea, called CPAP machines, can collect and report data through electronic means to the treatment provider. These means include, but are not limited to, automated data feeds facilitated through cloud based services and manually initiated methods such as data card downloading or analog telephone transmission. The near real time availability of this data allows a treatment provider to make decisions on the treatment and intervene according to a clinical protocol.

The data and accompanying management tools provided by CPAP device manufacturers are more than adequate to manage a small number of patients with a simplistic clinical protocol. However, the treatment provider is faced with several problems that have to be overcome in order to efficiently and effectively use the information provided by CPAP devices when there is a large number of patients under management, or the clinical protocols are complex.

The problems include multiple data sources from different manufacturers. For a large patient population, there tends to be a diversity of CPAP equipment from different manufacturers used for therapy. This creates different data streams origination from different cloud based services or different device types. The data will have different scales, different parameters, etc. and otherwise be non-uniform. In addition to the different data streams, manufacturers provide individual management portals, causing the provider to have to use multiple systems to manage a patient population.

Further, there is a lack of concise presentation for large data sets. The manufacturer management portals don't allow for customization of the presentation for large data sets, limiting the efficiently of managing a large population base. Further still, there is a lack of integration with a canonical patient database. Because the manufacturer management portals focus solely on CPAP data, they are unable to incorporate other types of patient information such as demographic, other clinical chart information, insurance, etc.

Another problem is a lack of analysis and presentation tools for complex clinical decision support. The manufacturer management portals focus on presenting information to manage to the Medicare definition of compliance. The data required for more sophisticated clinical protocols is not surfaced easily within their interface. Yet another problem is a lack of visibility to the patient. The manufacturer management portals target the health care provider as the end user and have no facility to display data to a patient. Providing the patient visibility to their usage and compliance to therapy may incentivize, or could be used in incentive programs to increase compliance.

### SUMMARY

Implementations of the current subject matter can include, but are not limited to, systems and methods consistent including one or more features are described as well as articles that comprise a tangibly embodied machine-readable medium operable to cause one or more machines (e.g., computers, etc.) to result in operations described herein. Similarly, computer systems are also described that may include one or more processors and one or more memories coupled to the one or more processors. A memory, which can include a computer-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including but not limited to a connection over a network (e.g. the Internet, a wireless wide area network, a local area network, a wide area network, a wired network, or the like), via a direct connection between one or more of the multiple computing systems, etc.

In one aspect, a system and method for managing treatment of a disorder of a person are disclosed. Data from multiple sources is collected in a data warehouse by a server computer. The data is associated with the disorder, and the sources including a clinical data source and a real-time monitoring source associated with the person. The collected data is normalized against a population of persons sharing the disorder. The normalized data is then associated with the person to generate a treatment regime of the disorder. Compliance by the person with the treatment regime is then calculated based on operational metrics for the population and the data collected from the plurality of sources.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to an enterprise resource software system or other business software solution or architecture, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 is a functional block diagram of a treatment management system.

FIG. 2 illustrates data sources for a treatment management system.

FIG. 3 illustrates a data input layer of a treatment management system.

FIG. 4 illustrates a data processing layer of a treatment management system.

FIG. 5 illustrates a data storage layer of a treatment management system.

FIG. 6 illustrates clinical and business logic of a treatment management system.

FIGS. 7-10B show various example reporting, tracking and management interfaces of a treatment management system.

### When practical, similar reference numbers denote similar structures, features, or elements.

### DETAILED DESCRIPTION

To address these and potentially other issues with currently available solutions, methods, systems, articles of manufacture, and the like consistent with one or more implementations of the current subject matter can, among other possible advantages, consolidate and structure disparate data from different data sources in a manner that allows all the data to interact with each other, and calculate metrics and values in a repeatable and automated manner. Further, the system can present data to multiple end users, including clinical staff, external physicians, patients, or other stakeholders for the purposes of reporting, decision support, operational support, and/or incentivization.

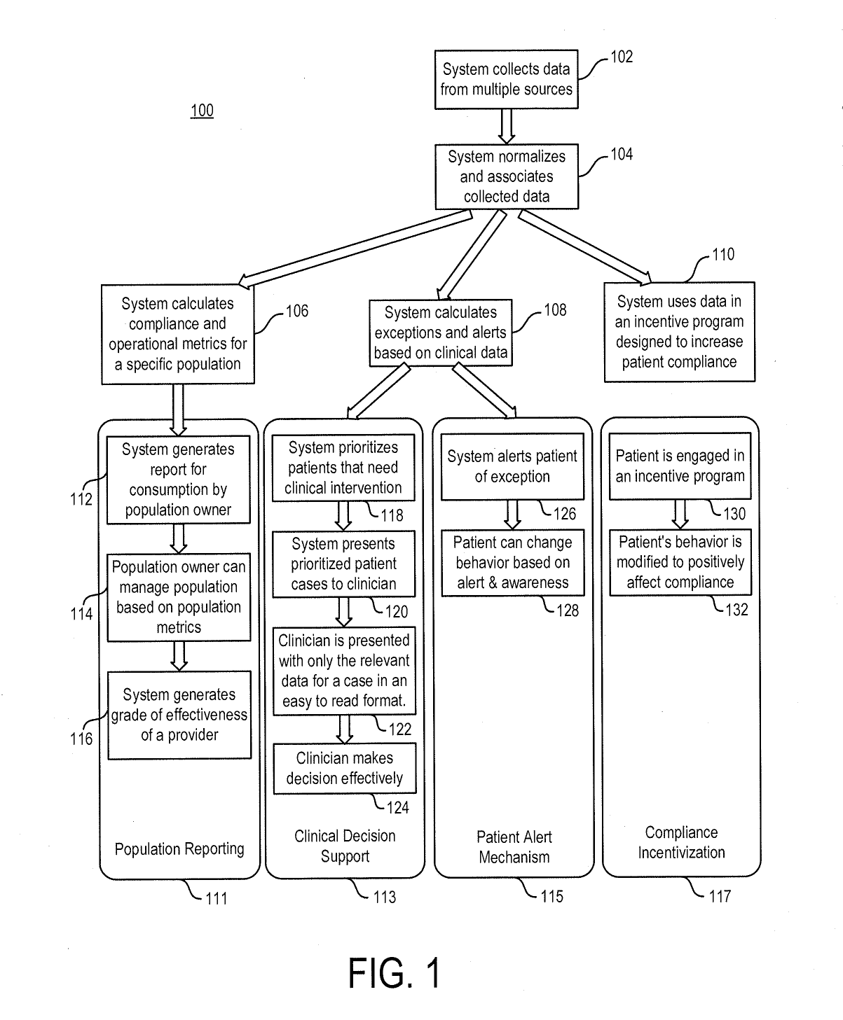

Multiple use cases are described below, with reference to FIG. 1, which illustrates a disorder treatment management process 100 of a disorder treatment management system. The process 100 includes collecting treatment data from multiple sources at 102. At 104, the system normalizes and associates the collected data. At 106, the system calculates compliance and operational metrics for a specific population. At 108, the system calculates exceptions and alerts based on clinical data, and at 110, the system uses data in an incentive program designed to increase patient compliance with treatment management. The process 100 further includes the following process modules:

The Population Reporting 111:

The system can provide screening, diagnostic and therapeutic services to a number of different entities. These entities include physician's practices, medical groups, employers, health care plans, and government institutions. The entities would like to have visibility into their patient's status along the care continuum and be able to use this information in their own interactions with the patient. At 114, these entities can define a population, and the population owner can manage the population based on population metrics. In addition, at 116 the entities can grade the system's performance based on population level metrics such as compliance and patient satisfaction. At 112, the system can aggregate all of the data required to support patient status reporting and population metrics for a given population and present it to the appropriate entity stakeholder.

Clinical Decision Support 113:

For the system to effectively manage a large patient based toward compliant behavior, processes and systems are required to identify where to deploy scarce clinical resources. The system gathers all clinical and operational data available for a patient from internal and external sources. At 118, after associating the data to a patient record, the system applies clinical algorithms to locate exceptional circumstances in the patient's diagnosis, treatment, or ongoing care that need to be addressed by a clinician. At 120, the system then highlights these exceptional patients to the clinician and presents only the relevant data, as pre-determined by clinician, so the clinician can make the appropriate clinician judgments based on the data presented, at 122. This targeted, push paradigm to manage only exceptional patients maximizes the clinician's efforts by allowing the clinician to focus only on the patients that need an intervention, and increases her productivity by gathering only the necessary information needed to make that decision and presenting it in an easily digestible format, at 124.

Patient Alert 115:

To date, patients have lacked feedback regarding how well they are doing on the prescribed therapeutic regimen. The patient can be associated with a program that allows the system to push notifications and alerts to the patient based on certain thresholds, at 126. Some of these alert threshold types are identified below. By alerting the patient, the patient can act to correct the situation before it becomes too severe, at 128.

Compliance Incentivization 117:

Unfortunately, even with alerts and notifications, patients may not comply with the therapeutic regimen. Compliance for treatment of chronic conditions averages 50% for most conditions, and CPAP treatment compliance is no different. In these cases, a higher level of interaction is required to change the behavior of the patient towards compliance. At 130, the system generates and deploys incentives as a mechanism to encourage changes in the patient behavior. The incentive can be financial, such as a discount on therapy supplies or medication, physical such as pens or other items, or virtual, such as an electronic badge or level stature. The incentives can also be structured in such a way as to make the act of compliance a game, where the patient is continually engaged and rewarded for achieving goals in a series of actions leading to or consistent with maintaining compliance, at 132.

The system includes a number of key functional layers, with accompanying functional modules within each layer providing robustness to the execution of the functional layer.

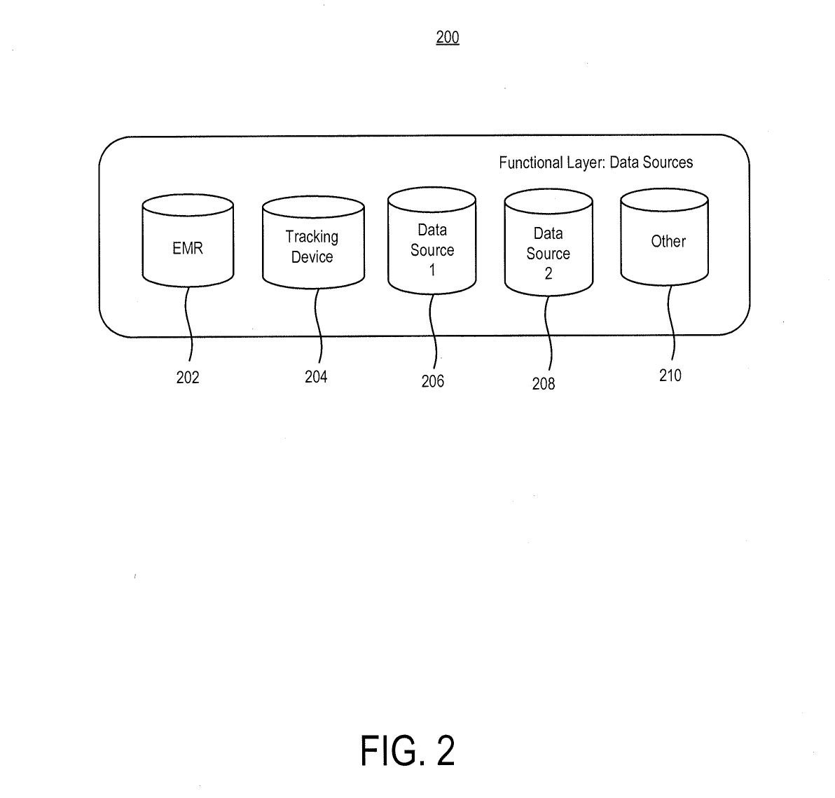

FIG. 2 illustrates a Functional Layer: Data Sources 200. This functional layer 200 includes originating data sources that the system accesses. The data sources may be internal to the system architecture, but can also be external or originate from one or more external software systems. The data sources can include the following:

Electronic Medical Records (EMR) System 202:

In one implementation, a cloud based EMR system provides data feeds through CSV reports that can be run on demand or on a schedule, an API, or through a periodic data warehouse download. The system can support other EMR's as the data source, especially if there are industry standard mechanisms that allow access to the data.

In some implementations, patient demographic data is imported from a CSV report that is downloaded on a periodic basis (nightly, every other day, etc.). The report contains patient information such as name, gender, birthdate, address, etc. In addition, the report can also contain information about the patient's physicians, insurance companies, and relatives.

The patient history is also stored in the EMR system and provided through a CSV report that is downloaded on a periodic basis (such as nightly). The patient history contains all of the notes created about the patient, including questionnaires, a call log, any clinical interactions, equipment configuration changes, etc.

Clinical state data, which details where a patient is within the clinical and operational workflow, is also extracted from a CSV report provided by the EMR system. For example, a patient state might be “DX-Patient Scheduled for PCP Visit” which would denote that the patient is currently in being scheduled for a visit to their primary care physician.

Tracking Device 204:

These could be wearable sleep tracking devices such as the Fitbit® or Jawbone® wearables or the like, or non-wearable devices such as the ResMed® S+ or Beddit® Sleep Monitor. This data source can provide tracking information relevant to the disorder being treated, such as a sleep tracking device.

Data Source 1 206, Data Source 2 208, and Other 210:

Positive Airway Treatment Devices:

Modern Positive Airway Treatment devices are equipped with cellular modems that transmit information from the therapy device to a service run by the device manufacturer. The device manufacturer in turn makes this information available to health care providers. Some of the information provided by the PAP devices includes time on treatment per night, treated apnea hypopnea index, mask leak, and device pressure.

Diagnostic Software:

Specialty devices, called sleep data recorders, are used to monitor physiological functions when a patient is sleeping for the purpose of diagnosing sleep apnea. Heart rate, respiratory effort, blood oxygen level, airflow, and movement are some of the functions monitored during the night. This data is collected for one or more nights of sleep and then analyzed by a sleep technician to determine how many times the patient has apneic events, a process called “scoring.” The scored data is then presented to a physician who then makes an interpretation of the data and diagnoses that patient as positive or negative for sleep apnea. The raw data, scored data, and interpretation data can be made available to other systems in text, csv, HTML, or other formats.

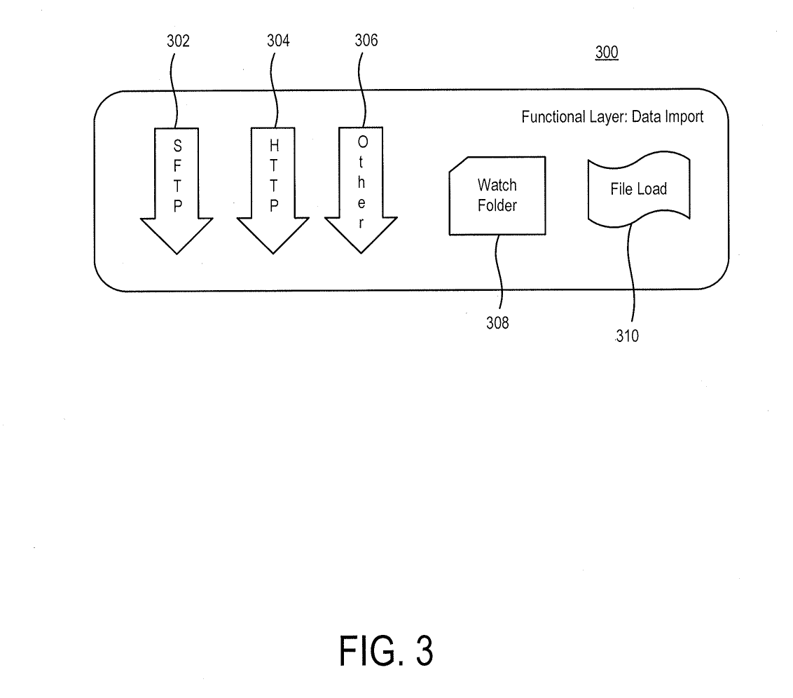

FIG. 3 illustrates a Functional Layer: Data Import 300:

In some implementations, the system can be architected to support multiple inbound data formats and structures, which may or may not be time sequenced, through a Data Import Layer. The layer supports the development of modules that interact with multiple external data feeds to import data in formats such as XML, HL7, CSV, JSON, HTML and other data formats. Custom data formats are also supported. Once the data is retrieved by the Data Import modules, it is sent to the Data Processing Layer.

Some of the Functional Modules for Data Import include the following:

Functional Module: External Site Download

The system downloads data from an external location, through a standard protocol such as SFTP 302, HTTPS 304, or other 306. This download, to achieve a file load 310, can occur automatically on a periodic basis, or be user initiated. The system performs error recovery if the import is unsuccessful.

Functional Module: Watch Folder 308

The system scans a local or network file folder location on an interval for new data to be imported. The system manages the process of importing the data, and performs error recovery if the import is unsuccessful.

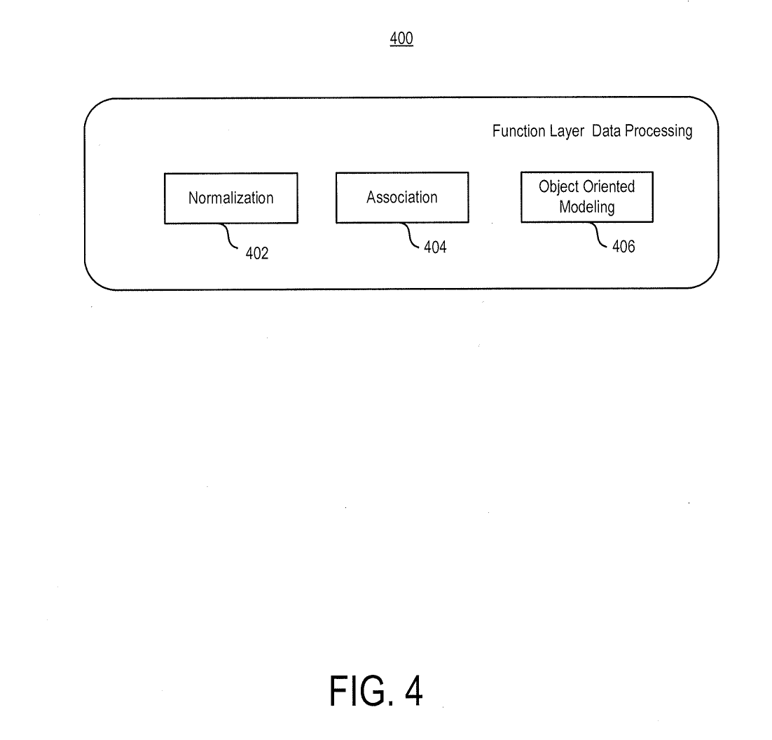

FIG. 4 illustrates a Functional Layer: Data Processing 400.

The Data Processing layer 400 manipulates imported data so it is baselined against a single standard and creates relationships between the data so that clinical and business logic can be easily applied to it.

It has three key functional modules; Normalization 402, Association 404, and Object Oriented Modeling 406.

Functional Module: Normalization 402

The system normalizes all of the data, with respect to units and type. For example, all data with duration information is normalized from hours or minutes to seconds, all timestamps are converted to UTC timestamps, and all units for a measurement (i.e., leak pressure) are converted to a standard unit (i.e., liters per minute) . In addition, records that are deemed invalid, or contain invalid data, are discarded.

Functional Module: Association 404

The system associates all imported data from different systems as appropriate. The association process creates linkages across disparate data sets and allows the end user to have a holistic view of an entity. One example of the association process is to link a patient demographic record from one system with his treatment usage data from a second system. More complex associations can be made across data sets.

Functional Module: Object Oriented Modeling 406

Object Oriented Modeling is the process of creating computing models that represent physical world objects or processes. This allows the data to be consolidated into objects that interact with each other, thereby reducing complexity when defining and implementing business or clinical logic in the system. The relationship between objects can be represented as an entity relationship diagram. Some of the entities and relationships that can be depicted are Patient, Sales Order, Doctors, Insurance, Sleep Study, etc.

In addition to the objects listed in the ERD, the following clinical process flow objects are also defined and associated with a patient: Screening, Diagnosis, Therapy, and OptOuts. Screenings are created when a prospective patient completes a screening instrument. This data is imported into the system manually or automatically through an API. Screenings record the screening date, basic patient demographic data (since patients at this stage are not always in the EMR), screener type, and a disease risk probability index based on an easy to use, low fidelity mechanism such as a survey.

Diagnoses are created from the results of a study, such as a sleep study or a study on insomnia. Diagnoses can be internal (performed by system) or external (from another clinic). Diagnoses can be of type: HST (Home Sleep Test) or PSG (PolySomnoGraph). Diagnoses also record the diagnosis date, clinical data, and outcome (positive or negative). Diagnoses also assign a severity index in the form of an AHI (apnea hypopnea index) reading.

Therapies are recorded when a patient who has been positively diagnosed gets setup with a course of treatment. As an example for a sleep disorder, therapies are of type: PAP (positive airway pressure) and its derivatives (CPAP, APAP, Bi-Level PAP) and MRD (mandibular repositioning device). PAP therapies have a prescribed pressure, setup date, and end date.

Patients may opt out of the clinical process at various points, e.g. they may decline a diagnosis (sleep study) after a positive screening or may decline treatment after a positive diagnosis. For reporting and business intelligence purposes, these events are modeled as OptOuts. OptOuts record the date opted out, the optout type, and allow for notes (e.g. reason if given). The stage at which a patient opted out of the process is determined by the system automatically via the presence and type of other objects in the system (e.g. given the presence of an optout object dated after a positive diagnosis object and without the presence of a therapy object, the system determines that the patient opted out of therapy).

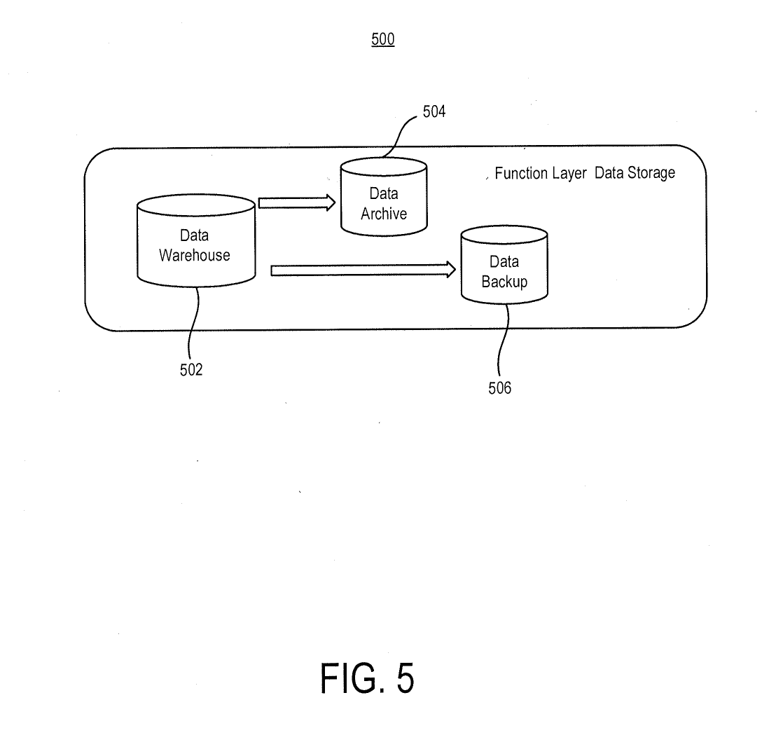

FIG. 5 illustrates a Functional Layer: Data Storage 500.

After the multiple, disparate data sources are imported and processed, the resulting data is stored in a central repository, or data warehouse 502. The data warehouse is a database that is used for reporting, clinical or business analysis, and other functions, such as patient incentivization.

The Data Storage layer also handles standard database management functionality such as backup, archival 504, performance optimization, etc. In one specific implementation, the Data Storage layer uses MySQL running on a Linux operating system as the database engine, or the like. The database undergoes continuous optimization at a software and hardware level to maintain an acceptable level of service and response times. Backups are performed through custom backup scripts for storage in a data backup 506.

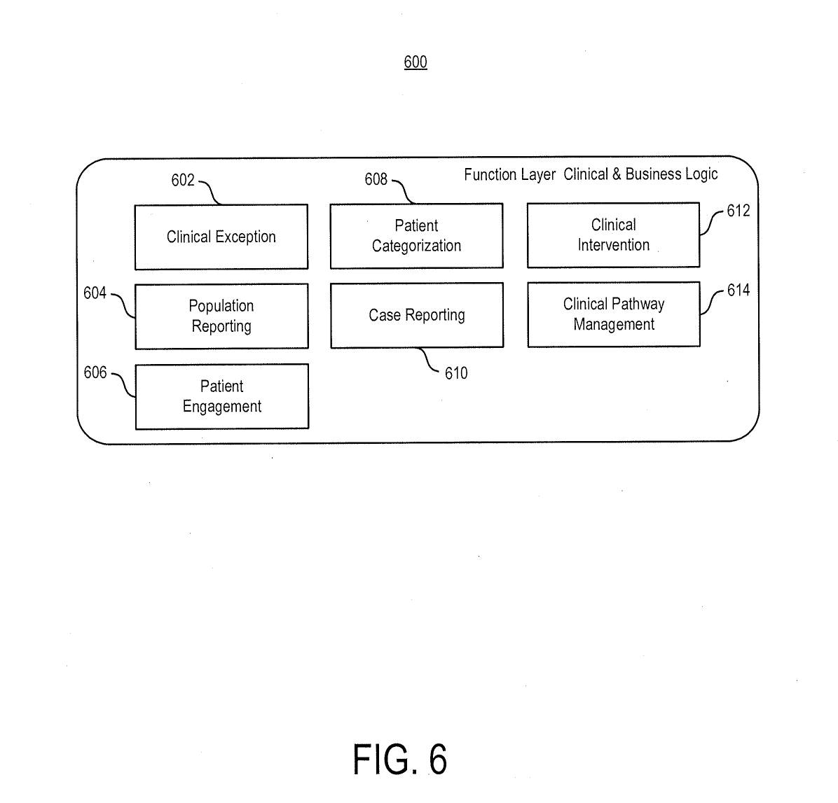

FIG. 6 illustrates a Functional Layer: Business and Clinical Logic 600

The Clinical and Business Logic layer 600 includes various functional modules to apply business analysis and clinical analysis to the processed dataset. This layer contains several modules, and can be extended over time to support the needs of the business, clinicians, and patients, and other stakeholders.

Functional Module: Clinical Exception 602

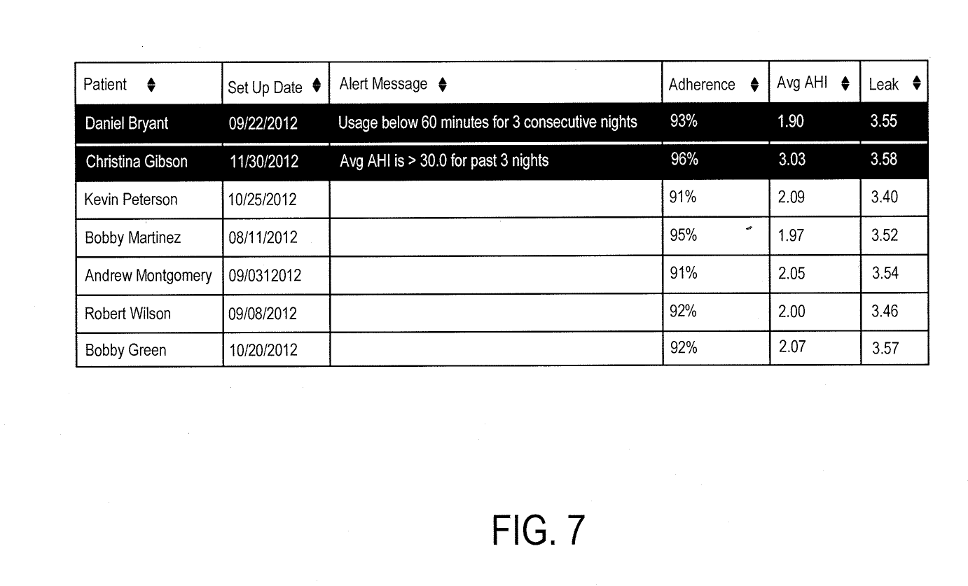

The Clinical Exception module 602 contains clinical intervention algorithms, which are applied to patient data in the system. The algorithms use the data values, trends within the data, and other data characteristics to identify the patients who most need an intervention in order to be successful on therapy. The Clinical Exception module 602 supplies this information in a near real time manner and allows the clinicians to focus their time on the patients who would most benefit from an intervention. A representation of a clinical exception dashboard is illustrated in FIG. 7, with exceptions highlighted and sorted to the top of the list.

The following is a list and description of exceptions that can be managed, in the context of sleep disorder treatment. It is representative and is not meant to be a complete, comprehensive list of exceptions, and other treatments can be managed by the Clinical Exception module 602.

Non-Usage:

If a clinician sees that a patient is not using his device (i.e. not adhering to the therapy regime) for three consecutive days, the clinician calls that patient and coaches the patient to use the device.

Low Usage:

If a clinician detects a pattern of low usage, i.e., the patient uses the device, but is using for less than the full amount of time that the patient is in bed, the clinician will call the patient to understand why the patient is removing the device in the middle of the night. Based on these reasons (visits to the bathroom, discomfort, nasal dryness, etc.) the clinician will coach the patient on techniques that increase the amount of time the patient wears the device during the night.

Intermittent Usage:

If a clinician sees a usage pattern that indicates intermittent usage, i.e., one night off, 4 nights on, 2 nights off, etc., the clinician will call or otherwise contact the patient to intervene and coach the patient to use the device on a regular basis.

High AHI:

AHI is an indicator of the severity of sleep apnea. While on treatment, the patient should have a treated AHI that is significantly lower than the untreated AHI. A clinician will intervene if a patient's treated AHI is not significantly lower than the non-treated AHI. This would signify that the current treatment regime is not effective in treating the condition and alternate therapy methods should be examined. This does not necessarily mean that the treated AHI falls within the bounds of a clinically insignificant AHI, i.e., less than 15.

A clinician might also intervene if she sees a trend where the treated AHI is increasing over time.

High Leak:

When the patient wears a CPAP mask for treatment, there should be a solid seal against the patient's face, and very little, if no leakage of air around the mask except for the normal expiratory ventilation when the patient exhales. If the data from a patient's CPAP device continually reports a high leak pressure (i.e., 24 liters per minute post normal ventilation leak) during a usage period, the clinician will intervene and contact the patient to coach the patient through the mask fitting process. The thresholds may be different values depending upon the therapy pressure and mask type.

Periodic breathing: If a clinician sees periodic breathing (breathing in abnormal periods, or with short, shallow breaths) greater than a threshold percentage (typically 10%), the clinician would intervene and determine the appropriate response based on the clinical circumstances.

Central Apnea Index:

If the patient has a central apneas index above 10 during PAP treatment, the clinician evaluates and determines the appropriate clinical course.

Blood Oxygen Level (SpO2):

If a clinician sees a hypoxemia (a decrease of the blood oxygen level to less than 90%), the clinician will intervene and respond accordingly based on the clinical circumstances.

Respiratory Rate:

If a clinician sees a respiratory rate that is higher (hyperventilation) or lower (hypoventilation) than normal for the patient, the clinician would intervene accordingly based on the clinical circumstances.

Heart Rate:

If the clinician sees a heart arrhythmia (tachycardia, bradycardia, irregular rhythm) this might indicative of respiratory distress in the patient and can act as a accelerator to have the patient visit a specialist or their primary care physician.

Functional Module: Patient Categorization 608

The Patient Categorization module groups patients into specific groups based on their behaviors, demographics, and other assessments for the purposes of identifying the most effective treatment protocol for the patient or to report against a specific patient cohort. By grouping patients into a limited number of categories, the clinicians are able to reduce the number of different treatment protocols that have to be supported.

Categorizations:

Adherent/Non-Adherent:

Within the Positive Air Pressure (PAP) therapy regime, adherent patients are those that are using their device for at least 4 hours for 70% or more of the nights in the time period under evaluation. For example, if the patient's adherence for the last ten nights of usage are being calculated, and the patient used the device for six hours on six of the ten nights, and three hours on the remaining four nights, he would have an adherence value of 60% and be non-adherent because he did not reach the 70% threshold. Operationally, clinicians will sort a patient list by adherence values and use this information to schedule follow up actions as necessary. For example, the group of patients who are 85% to 100% adherent may be called for a follow up once a quarter at minimum, whereas patients who are 75% to 85% adherent may be called once a month at minimum. Patients who are less than 70% adherence may receive a bi-weekly follow up call and so on.

Compliant/Non-Compliant:

Compliance is built on top of adherence by setting specific timeframes to be adherent. The compliance measurement is used by Medicare and other payors to determine the effectiveness of a provider. Being compliant is defined as having a 30 day time period, within the first 90 days of therapy, where the patient is adherent. In practice, this results in a patient having twenty one nights within a thirty period, during the first ninety days of treatment, where the patient uses the PAP device for more than four hours a night. The compliance rate is simply the percentage of patients in a measurement group that are compliant. Due to payment incentives for compliant patients and the significance of compliance rate as a competitive differentiator, the patient's usage is tracked very closely during the first ninety days and effort is applied to drive a patient towards a usage pattern that will result in a compliant patient. Patients that are close to being compliant and still have days remaining in the ninety day window typically get more attention than those who have already complaint or are non-compliant.

Usage Based Categorizations:

Patients can also be categorized into groups based on their PAP usage. In some implementations, patients can be categorized into multiple categories, such as seven specific categories. Specific treatment and intervention protocols can be developed for these categories to more efficiently and effectively deliver treatment to the patient population.

Functional Module: Clinical Intervention Protocols 612

The Clinical Intervention Protocols module contains the methodologies, processes, and tracking mechanisms for intervening into a patient's treatment regimen.

Functional Module: Population Reporting 604

The Population Reporting module aggregates data, calculates metrics, and where appropriate, de-identifies confidential patient data to generate aggregate or population level reports. It tracks patients as they go through the Patient Lifecycle, which is composed of screening, diagnosis, treatment, and ongoing care.

Population Funnel:

This report details how many patients are in each phase of the Patient Lifecycle and details their status accordingly. The status can be one of the following:

Inactive/Unable to Contact—The patient has been unreachable for a certain period of time, or a certain number of reach out attempts. The current threshold to enter this state is 6 unsuccessful email or phone calls in a month period, but the thresholds may be altered depending on the implementation.

Scheduling—The patient is working to book an appointment, or has an appointment that has not yet occurred with a health care provider, typically via the system or their primary care physician (PCP).

Negative—The patient does not meet the requirements for a positive result for the particular phase of the Patient Lifecycle.

Positive—The patient meets the requirements for a positive result for the particular phase of the Patient Lifecycle.

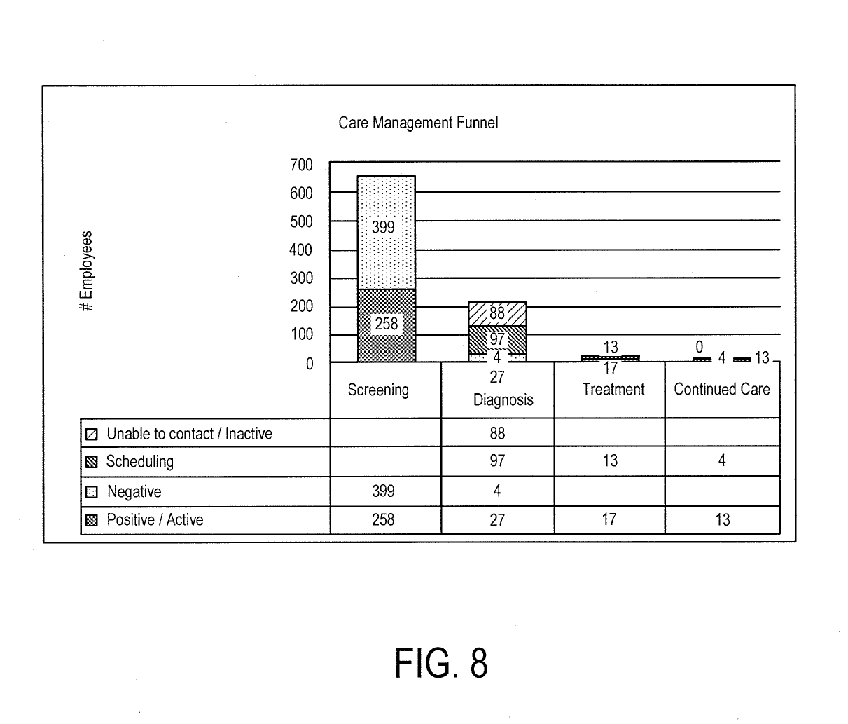

See the example provided in FIG. 8.

Other population level reports might detail the prevalence of the disease in the population, the type of disease, the type of diagnosis type, the treatment participation level, usage, adherence, and treatment types. However, this list should not be construed as an inclusive list of all population level reports. Some samples are provided below.

Functional Module: Case Reporting 610

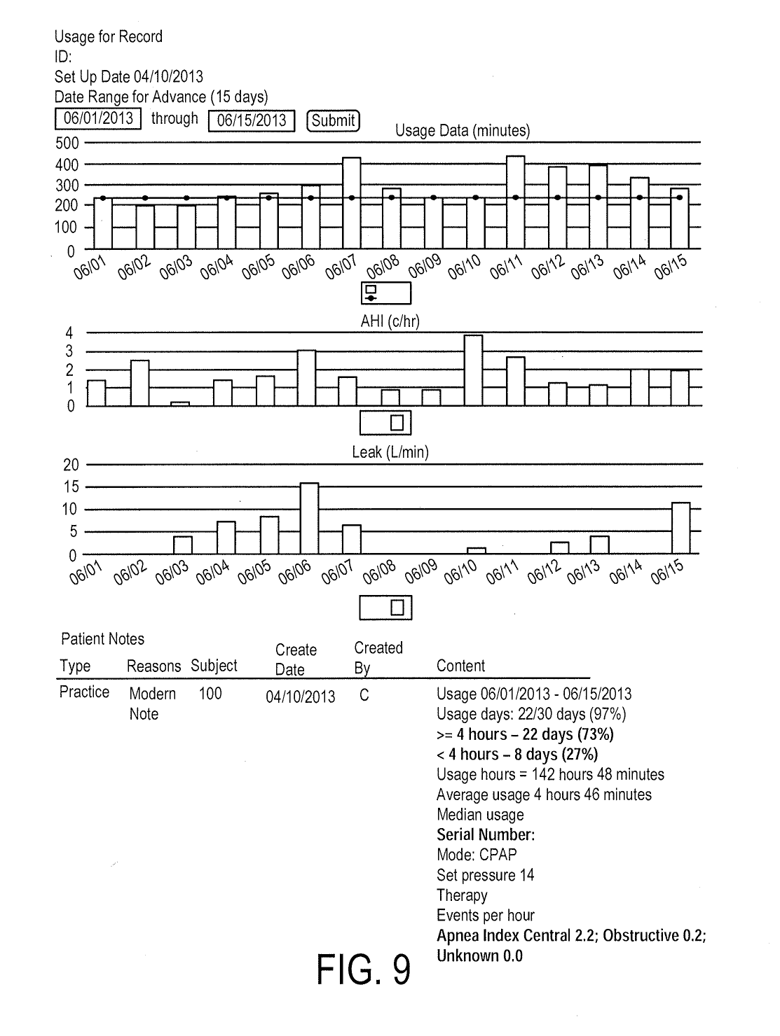

The Case Reporting module allows the system user to obtain data and reports about a specific patient case after aggregating the information from the different data sources. The Case Reporting module also calculates statistics for individual patient level analysis. This can be presented in tabular or other sortable format, as illustrated in FIG. 9. In addition, a patient's clinical data, gathered from the PAP device can be shown along with the patient's chart notes.

Functional Module: Clinical Pathway Management 614

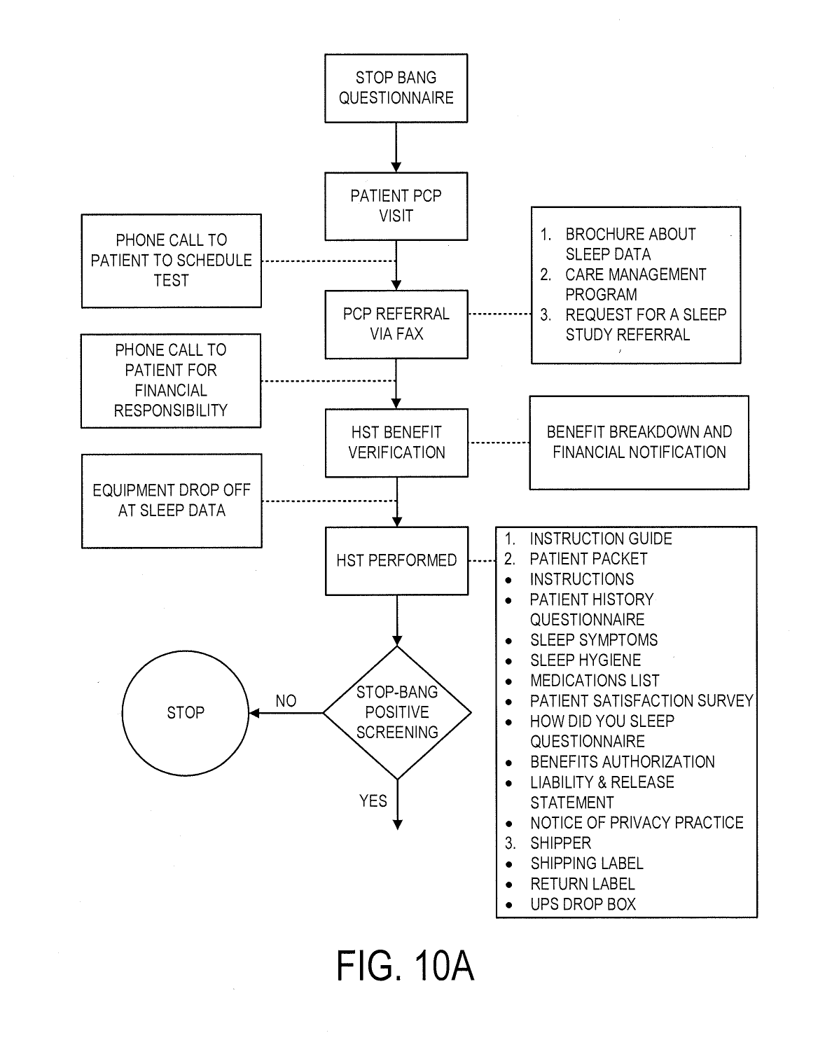

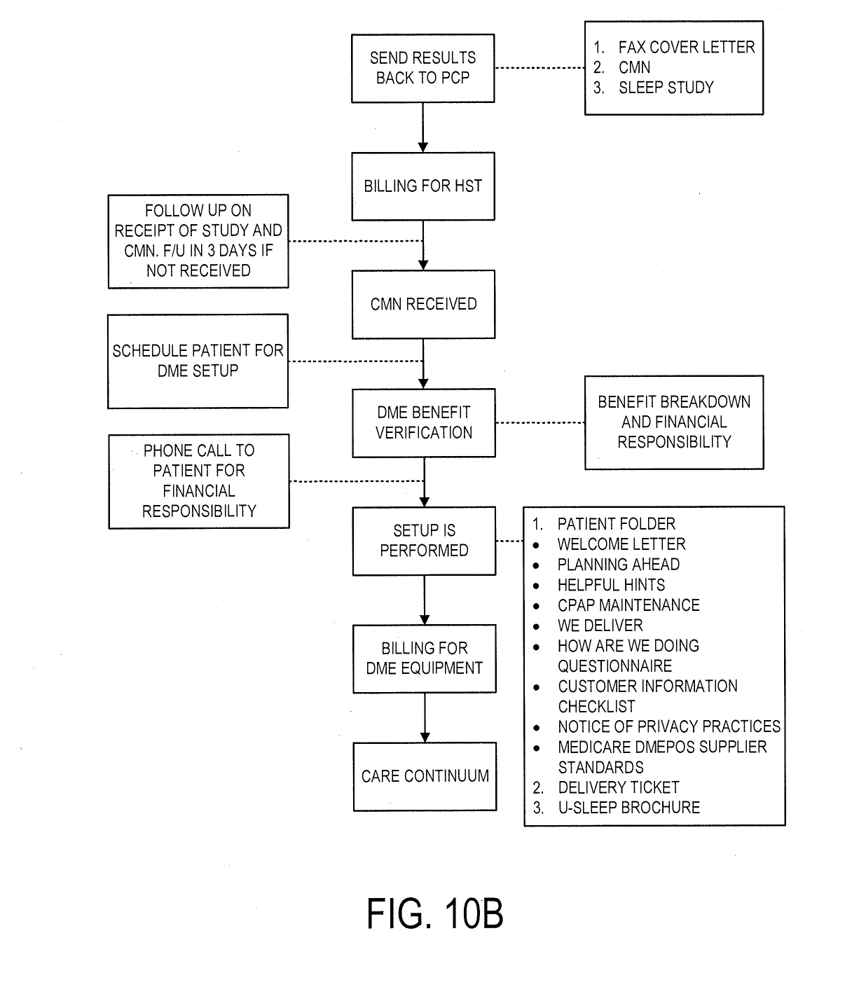

The Clinical Pathway Management module 614 manages the clinical workflow and ensures that patients are moved efficiently from one process to another. It achieves this by ensuring that prerequisites are met before the patient moves to the next step and alerting users when the process flow continuation conditions are not met. FIGS. 10A and 10B illustrates a sample patient process flow diagram for some of the conditions as generated by the system.

Functional Module: Patient Engagement 606

The Patient Engagement module 606 includes and executes one or more algorithms to categorize, analyze, and present activities to a patient that would modify their behavior in a specific manner. Some implementations include the following engagement mechanisms:
Alert display/email/text/call or other reachout when usage falls below a certain level;
Encouragement display/email/text/call or other reachout when a patient meets a certain level of usage;
Encouragement display/email/text/call or other reachout when a patient is borderline towards reaching a goal;
Encouragement display/email/text/call or other reachout on a random interval based on positive behaviors;
Gamification for the purposes of encouraging therapy usage;
Points given for discrete, small goals or milestones;
Badges earned for meeting consecutive goals or attaining a certain level;
Incentives, which can be monetary, physical goods, services, or virtual goods for certain behaviors or attainment of specified goals.

The system can engage the patient over a variety of channels, such as an automatically generated phone call, regular mail, email, newsletter, text, website, mobile application, personal computer application, wearable device, etc.

Functional Layer: Presentation Mechanisms

This functional layer includes the mechanisms for interacting with the system from an end user perspective, whether that end user is a clinician, referring physician, patient, or other stakeholder. The mechanisms can include, without limitation: Tablet/Mobile device; Personal Computer; Email; Voice; Wearable Technology

As an example, in diagnosing sleep apnea, the typical practice is to do a multi-night study; i.e., the patient is observed for more than one night. The first night is used to diagnose whether the patient has sleep apnea or not. If the patient is positively diagnosed for sleep apnea, the consequent night is used to determine a treatment pressure for PAP therapy in a process called titration. PAP therapy can be considered as “an airway splint” that keeps the airway open through the use of air pressure. It is critical the pressure of the air in PAP therapy be set properly according to the patient's circumstances. Ideally, the lowest pressure that keeps the airway open is prescribed.

The system can be configured to provide a process of only having a patient sleep with a recorder for one night and determining the PAP pressure formulaically through certain variables that describe the patient (BMI, neck diameter, untreated AHI). This halves the time needed for a diagnosis, allowing higher utilization of the expensive sleep recorder devices, reduces sleep study re-take rates, and increases the convenience for the patient.

The system can also be configured to provide a prescription that allows clinical latitude in setting the PAP pressure by +/−2 cmH2O for a patient. This allows for immediate intervention without having to wait for a new prescription.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein can be implemented on a computer having a display device, such as for example a cathode ray tube (CRT), a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A method of managing treatment of a disorder of a person, the method comprising:
collecting, in a data warehouse by a server computer, data from a plurality of sources, the data being associated with the disorder, the plurality of sources including a clinical data source and a real-time monitoring source associated with the person;
normalizing, by the server computer, the collected data against a population of persons sharing the disorder;
associating, by the server computer, the normalized data with the person to generate a treatment regime of the disorder; and
calculating, by the server computer, a compliance by the person with the treatment regime based on operational metrics for the population and the data collected from the plurality of sources.

2. The method in accordance with claim 1, further comprising calculating exceptions and generating alerts based on data from the clinical source that is outside a range established by the normalized data.

3. The method in accordance with claim 1, further comprising generating, by the server computer, a graphical report for transmission to a client computer and display on the client computer.

4. A non-transitory computer program product storing instructions that, when executed by at least one programmable processor, cause the at least one programmable processor to perform operations comprising:
collecting, in a data warehouse, data from a plurality of sources, the data being associated with a disorder, the plurality of sources including a clinical data source and a real-time monitoring source associated with a person having the disorder;
normalizing the collected data against a population of persons sharing the disorder;
associating the normalized data with the person to generate a treatment regime of the disorder; and
calculating a compliance by the person with the treatment regime based on operational metrics for the population and the data collected from the plurality of sources.

5. A system comprising:
at least one programmable processor; and
a machine-readable medium storing instructions that, when executed by the at least one processor, cause the at least one programmable processor to perform operations comprising:
collecting, in a data warehouse, data from a plurality of sources, the data being associated with a disorder, the plurality of sources including a clinical data source and a real-time monitoring source associated with a person having the disorder;
normalizing the collected data against a population of persons sharing the disorder;
associating the normalized data with the person to generate a treatment regime of the disorder; and
calculating a compliance by the person with the treatment regime based on operational metrics for the population and the data collected from the plurality of sources.
